(19) United States
(12) Patent Application Publication
(10) Pub. No.: US 2018/0242889 A1
(43) Pub. Date: Aug. 30, 2018

(54) METHOD FOR MEASURING AMOUNT OF MOVEMENT OF ANIMAL

(71) Applicant: A&D COMPANY, LIMITED, Tokyo (JP)

(72) Inventors: Naoto IZUMO, Saitama (JP); Ken IHARA, Saitama (JP)

(21) Appl. No.: 15/754,619

(22) PCT Filed: Oct. 9, 2015
(86) PCT No.: PCT/JP2015/078759

§ 371 Date: Feb. 23, 2018

(51) Int. Cl. A61B 5/11; A61D 3/00; G01G 19/414

(52) U.S. Cl. CPC A61B 5/1126; A61D 3/00; A61B 2503/42; A61B 2503/40; G01G 19/414

(57) ABSTRACT

Provided is a new method of measuring an amount of movement of an animal. A measurement value of an animal as a measuring target is sequentially measured with a scale (21), an amount of change in the measurement value obtained in chronological order is calculated, and from division of the amount of change in the measurement value by a weight of the animal, an amount of movement of the animal is measured. Accordingly, weight differences between individual living bodies and in the process of breeding are eliminated, and an amount of movement of an animal based on changes in the weight of the living body can be measured.

### TECHNICAL FIELD

The present invention relates to a method of measuring an amount of movement of an animal in an animal experiment, etc.

### BACKGROUND ART

In an animal experiment, in order to observe an effect of a dose of a medical agent or toxic agent, generally, the weight of an animal is continuously measured. An increase or decrease in weight is an effective factor for evaluation of a physical condition of a living body, and in recent years, there is a demand for measurement of a degree of activity of a living body by quantifying an amount of movement of an animal in some way.

Well-known methods of quantifying an amount of movement of an animal are:

(1) an optical system in which a light path is determined and a light emitting element and a light receiving element are disposed, and the number of passages of an animal through the light path is measured (Patent Literature 1),

(2) a method in which the whole of a cage in which an animal is kept is imaged by a CCD camera, and the numbers of entries of the animal into the respective areas set in the cage are measured (Patent Literature 2),

(3) a method in which an RFID tag is attached to an animal, and a movement locus is measured based on positional information of an RFID reader (Patent Literature 3), and

(4) a method in which a rotary basket is put in a cage, and the number of rotations of the rotary basket is measured.

(5) Besides these, there is a method in which an animal is placed on a measuring platform of a balance, a measurement value of the animal is acquired by the balance for a set time, and absolute values of differences between static loads of the animal and the measurement values are temporally integrated, and the integrated value is used as an amount of activity of the animal (Patent Literature 4).

### CITATION LIST

### Patent Literatures

Patent Literature 1: Japanese Published Unexamined Patent Application No. H07-184515

Patent Literature 2: Japanese Published Unexamined Patent Application No. H08-32959

Patent Literature 3: Japanese Published Unexamined Patent Application No. 2002-58648

Patent Literature 2: Japanese Published Unexamined Patent Application No. H06-133956

### SUMMARY OF THE INVENTION

### Technical Problem

However, data obtained by the methods (1) to (4) described above are digital counts including the number of times of coming and going of an animal and the number of rotations of the basket, and it is difficult to consider physical amounts that the count numbers mean. The method (5) described above has a problem in which obtained data is a measurement value measured on the measuring platform, so that spilled feed or spilled water may influence the amount of movement, and when the weight of the animal increases with its age in weeks, a measurement value change becomes larger after the increase in weight even if motion is the same, and therefore, the amount of movement is evaluated to be larger.

An object of the present invention is to solve the above-described problem, and provide a new method of measuring an amount of movement of an animal.

### Solution to Problem

In order to solve the above-described problem, a method of measuring an amount of movement of an animal according to an aspect of the present invention includes: sequentially measuring a measurement value of an animal as a measuring target by a scale, calculating an amount of change in the measurement value obtained in chronological order, and measuring an amount of movement of the animal by division of the amount of change in the measurement value by a weight of the animal.

A method of measuring an amount of movement of an animal according to an aspect of the present invention includes: sequentially measuring a measurement value of an animal as a measuring target by a scale, setting a predetermined calculation interval of the amount of movement, calculating a difference between a latest measurement value and a previous measurement value, calculating an integrated value by integrating absolute values of the differences, and calculating, at the calculation intervals of the amount of movement, a value by dividing the integrated value by an average value of weights of the animal in the calculation interval of the amount of movement or a weight of the animal at a point of time in the calculation interval of the amount of movement, and defining the value as an amount of movement of the animal.

In the aspect described above, it is also preferable that the amount of movement is visualized and output by taking a time of calculation of the amount of movement on one axis and the amount of movement on the other axis.

In the aspect described above, it is also preferable that as the measurement value, a value acquired in a state where a weighing pan of the scale is disposed inside a breeding container of the animal is used.

In the aspect described above, it is also preferable that a weight of the animal is determined by a difference between a measurement value when the animal is judged to have gotten onto the weighing pan of the scale and a measurement value when the animal is judged to have gotten off the weighing pan.

### Advantageous Effects of Invention

According to the present invention, a scale that sequentially measures a measurement value is used, and a value the unit of which is made dimensionless by division of an amount of change in measurement value by a weight of an animal calculated from the measurement value, is newly defined as an amount of movement. Thereby, weight differences among individual living bodies and in the process of breeding are eliminated, and an amount of movement of an animal based on changes in weight of the living body can be measured.

### BRIEF DESCRIPTION OF DRAWINGS

FIG. 1 is a configuration block diagram of an amount of movement measurement system for an animal, according to an embodiment.

FIG. 2 is a right side view of an animal weighing scale to be used in the system of FIG. 1.

FIG. 3 is a flowchart of a method of measuring an amount of movement of an animal according to the embodiment.

FIG. 4 is a flowchart of a method of measuring a weight in the flowchart of FIG. 3.

FIG. 5 is a graph visualizing an amount of movement obtained in the embodiment.

FIG. 6 is a graph visualizing an amount of movement obtained in the embodiment.

FIG. 7 is a diagram showing a difference between an amount of movement obtained in the embodiment and an amount of movement obtained in a comparative example.

### DESCRIPTION OF EMBODIMENTS

Next, preferred embodiments of the present invention are described based on the drawings.

### (System Configuration)

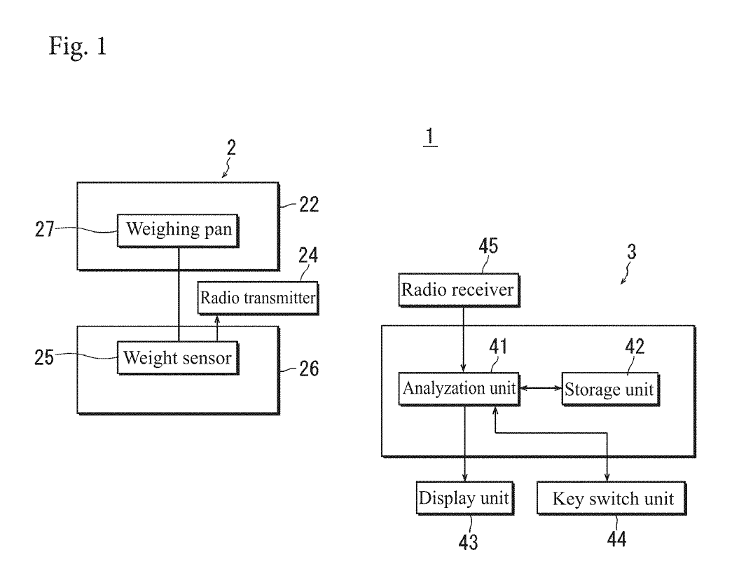

As shown in FIG. 1, an amount of movement measurement system 1 for an animal (hereinafter, simply referred to as a system 1) of the present embodiment includes an animal weighing scale 2 and an analyzation equipment 3. The animal weighing scale 2 preferable for the system 1 includes, as described below, a weighing pan 27 disposed inside a breeding container 22 (inside a breeding space 20), and a weight sensor 25 disposed outside the breeding space 20.

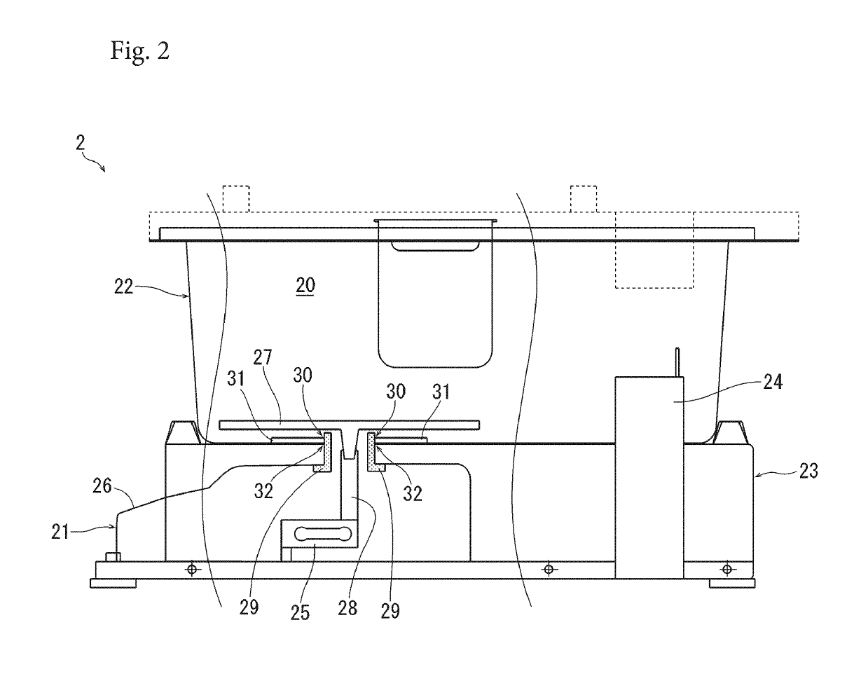

The animal weighing scale 2 includes, as shown in FIG. 2, a scale 21 (balance), a breeding container 22 (breeding cage), a support case 23, and a radio transmitter 24. In FIG. 2, for understanding of the configuration of the disposition of the weighing pan 27, the scale 21 is shown in section.

The scale 21 includes a main body case 26 with a built-in weight sensor 25, the weighing pan 27, a pan supporting post 28, and a circumferential wall 29. The weight sensor 25 may be of an electromagnetic balance type, a strain gauge type, a capacitance type, etc., and acquires measurement data of an object placed on the weighing pan 27. In place of the weighing pan 27, play equipment for exercise of an animal or a nest box for rest of an animal can also be used. As the weight sensor 25, an appropriate one may be selected depending on requirements of a weighing capacity, a minimum display (measurement value reading accuracy), and strength performance corresponding to a weight of an animal as an object of experiment.

The pan supporting post 28 is a hollow member that joins the weighing pan 27 and the weight sensor 25, and is fixed to the weight sensor 25 and extends upward in the vertical direction from the weight sensor 25. The pan supporting post 28 has a necessary length (height) to cause the weighing pan 27 to project to the inside of the breeding container 22. The circumferential wall 29 consists of a hollow portion surrounding in the circumferential direction the pan supporting post 28 projecting from the main body case 26, and a base portion of the hollow portion, and is fixed to an upper surface of the main body case 26.

During an experiment, an animal is kept inside the breeding container 22 (breeding space 20). In the bottom surface of the container, a bottom surface opening 30 to allow passage of the pan supporting post 28 and the circumferential wall 29 is formed. Between the circumferential wall 29 and the bottom surface opening 30, a diaphragm 31 to eliminate a gap is disposed.

The breeding container 22 is supported from below by the support case 23. The support case 23 has an opening on the front side, and from this opening, the scale 21 can be operated. In the upper surface of the support case 23, a case hole 32 to allow passage of the pan supporting post 28 and the circumferential wall 29 is formed. The breeding container 22 is positioned by the circumferential wall 29, and the total weight of the breeding container 22 is supported by the support case 23. Therefore, all weights including the weight of the breeding container 22 itself and weights of feed, water, and breeding papers, etc., are received by the support case 23, and weights other than objects placed on the weighing pan 27 are not weighed by the scale 21.

In the support case 23, a radio transmitter 24 is installed. Measurement data detected by the weight sensor 25 is converted into a measurement value by a CPU inside the scale 21, output to the radio transmitter 24 via an RS-232C cable, and received by a radio receiver 45 on the analyzation equipment 3 side described below.

With this configuration, in the system 1, it is not necessary to take an animal as a measuring target out of the breeding container 22, and the weight thereof can be measured while the breeding environment is maintained. As a modification of the present embodiment, the weight sensor 25 may also be disposed inside the breeding space 20. Details of the modification are described in International Application No. PCT/JP2015/62508 applied by the applicant of the present application.

Next, the analyzation equipment 3 is a PC (personal computer) which may be a general-purpose one including an analyzation unit 41 including a CPU, a ROM, and a RAM, etc., a storage unit 42 consisting of a magnetic hard disk, a semiconductor memory, etc., a display unit 43, and a key switch unit 44, etc. An experimenter can perform various operations from the key switch unit 44, and can check various operations and analyzation results on the display unit 43. To the analyzation equipment 3, the radio receiver 45 is connected. A signal of a measurement value received by the radio receiver is sequentially recorded in the storage unit 42 in association with time. In the storage unit 42, various programs to perform flowchart processings described below are stored, and the analyzation unit 41 executes the programs.

(Method of Measuring Amount of Movement)

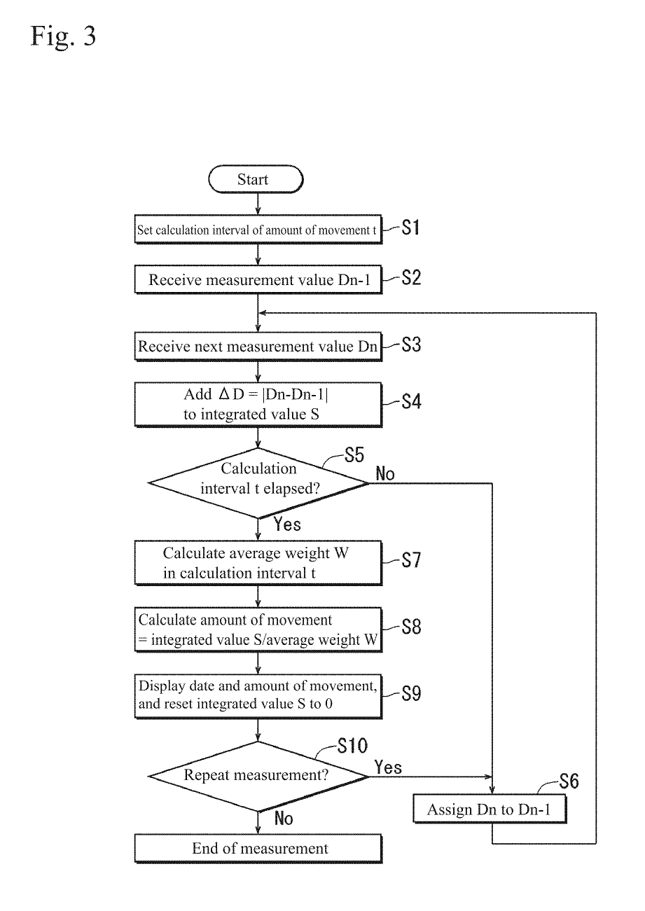

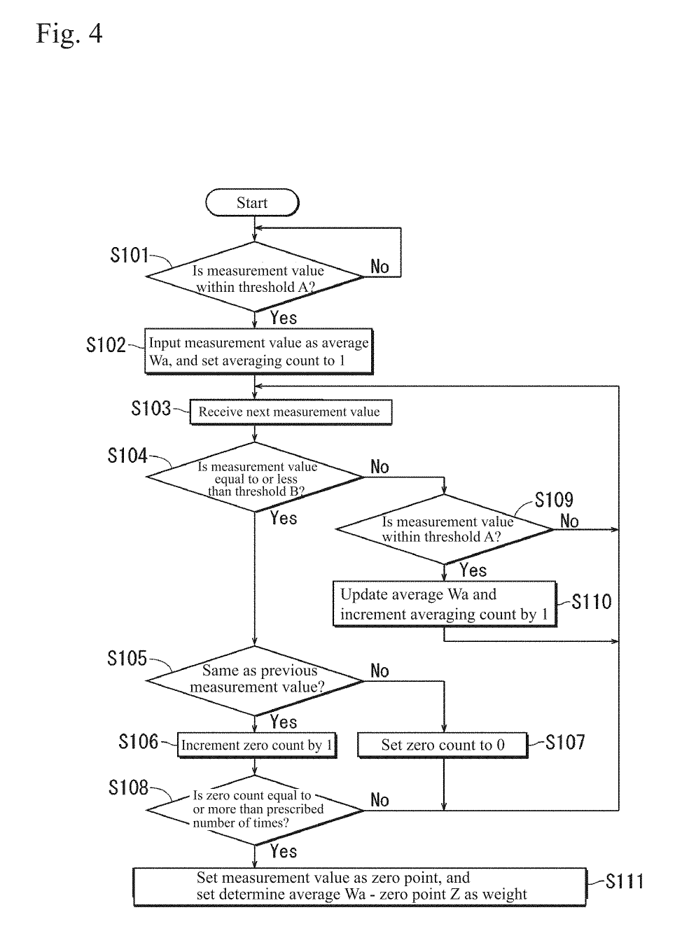

Next, a method of measuring an amount of movement of an animal, to be performed in the system 1, is described based on the flowchart of FIG. 3. The following are defined in order to avoid misunderstanding in the following description, “measurement value” is a value (raw data) obtained by converting measurement data acquired by the weight sensor 25 into a measurement value, and “weight” means a “weight value” determined in the flowchart of FIG. 4 described below.

First, in Step S1, a calculation interval of the amount of movement t (hereinafter, simply referred to as a calculation interval,) at which an amount of movement is calculated is arbitrarily set from the key switch unit 44. The calculation interval t is preferably, for example, 1 hour for sequential measurement to be continued for several weeks, 30 minutes for sequential measurement to be continued for several days, and 10 minutes for sequential measurement to be continued for several hours, and so on.

Next, the process shifts to Step S2, and a measurement value Dₙ₋₁ measured on the weighing pan 27 (n indicates the number of times of sampling) is received.

Next, the process shifts to Step S3, and a measurement value Dₙ measured on the weighing pan 27 is received. As acquisition of a measurement value, for example, a measurement value is sampled about 10 times per second.

Next, the process shifts to Step S4, and an absolute value of a difference ΔD between the measurement value Dₙ and the previous measurement value Dₙ₋₁ is added to an integrated value S of prior differences (integrated value S=∫ΔD). When the animal performs an action such as getting onto the weighing pan 27, pushing the pan, jumping on the pan, etc., the measurement value changes, so that by always integrating changes in measurement value (differences ΔD), an amount of activity can be measured. In order to prevent the amount of activity from decreasing when the difference ΔD becomes negative, it is essential to use an absolute value (ΔD=|Dₙ-Dₙ₋₁|).

Next, the process shifts to Step S5, and whether the calculation interval t has elapsed is judged.

When the calculation interval t has yet to elapse, the process shifts to Step S6, the value Dₙ is assigned to Dₙ₋₁, and then, the process returns to Step S3 and repeats integration. When the calculation interval t elapses, the process shifts to Step S7, and an average weight W of the animal in the calculation interval t is obtained. The method of calculating the weight to be used in Step S7 is described below.

Next, in Step S8, the integrated value S obtained in Step S4 is divided by the average weight W obtained in Step S7, and a resultant value is calculated as an amount of movement of the animal (amount of movement=S/W) and saved together with the date and time of the calculation.

Next, in Step S9, the date and time and the amount of movement obtained in Step S8 are displayed on the display unit 43, and the integrated value S is reset to zero.

Next, in Step S10, whether repetition of the measurement is to be continued is judged. When the measurement is continued, the process shifts to Step S6, and calculation of the amount of movement is repeated. When the measurement is not continued, the measurement ends.

In the flowchart described above, in Step S7, an average weight of weights acquired for the whole time of the set calculation interval t is calculated, and in Step S8, a value obtained by dividing the integrated value S by the average weight W is defined as an amount of movement. In place of this average weight W, (A) an average value of weights acquired for a time as a part of the calculation interval t, or (B) a weight at a point of time in the calculation interval t, may be used. In detail, in the case of (A), when the calculation interval is 24 hours, in Step S7, an average value Wp of weights obtained during a period until one hour before the end time is obtained, and a value obtained by dividing the integrated value S by this average weight Wp, may be defined as an amount of movement. In the case of (B), in Step S7, a weight w when the calculation interval t elapsed is obtained, and a value obtained by dividing the integrated value S by this weight w may be defined as an amount of movement. In each case, the calculation time in Step S7 can be shortened.

Next, based on the flowchart of FIG. 4, a preferable method of measuring a weight of an animal in Step S7 described above is described. Details of this method are described in International Application No. PCT/JP2015/65598 filed by the applicant of the present application, so that only an essential point is described here.

A weight of an animal is calculated in Step S7, and this weight is determined by a difference between a measurement value when the animal is judged to have gotten onto the weighing pan 27 and a measurement value when the animal is judged to have gotten off the weighing pan 27.

First, in Step S101, the analyzation equipment 3 judges whether a sampled measurement value Dₙ is within the range of a threshold A. When the value is not within the range of the threshold A (No), a next measurement value is received. When the value is within the range of the threshold A (Yes), the process shifts to Step S102.

Next, in Step S102, the measurement value Dₙ of Step S101 is set as a measurement average Wa and the averaging count is set to 1, and then, the process shifts to Step S103. In Step S103, when a next measurement value Dₙ₊₁ is received, the process shifts to Step S104.

In Step S104, whether the measurement value Dₙ₊₁ of Step S103 is equal to or less than the threshold B (B<A) is judged. When it is equal to or less than the threshold B (Yes), the process shifts to Step S105. When it is more than the threshold B (No), the process shifts to Step S109.

In Step S105, whether the measurement value Dₙ of Step S103 is comparable with the previous measurement value Dₙ₋₁ (for example, within ±0.01 g of the previous measurement value) is judged. When it is comparable with the previous value (Yes), the process shifts to Step S106, the zero count is incremented by 1, that is, the count number of matches is incremented, and the process shifts to Step S106. When the value is not comparable with the previous value (No), the process shifts to Step S107, and the zero count is set to 0, that is, the number of matches is reset, and then the process returns to Step S103.

In Step S108, whether the zero count counted in Step S106 has reached a prescribed number of times (a fixed time, for example, equivalent to two seconds) or more, is judged. When it is less than the prescribed number of times (No), the process returns to Step S103. When it is equal to or more than the prescribed number of times (Yes), the process shifts to Step S111.

On the other hand, when the process shifts from Step S104 to Step S109, whether the measurement value Dₙ₊₁ is within the range of the threshold A is judged again. When it is not within the range of the threshold A (No), the process returns to Step S103. When it is within the range of the threshold A (Yes), the process shifts to Step S110.

In Step S110, when the difference between the measurement value Dₙ₊₁ and the measurement average Wa is equal to or less than a predetermined stable width C (for example, 2% of Wa), the measurement average Wa is updated by adding the measurement value W, and the averaging count is incremented by 1. Then, the measurement average Wa and the averaging count at this time are updated, and the process returns to Step S103.

When the process shifts to Step S111, a measurement value whose number of matches is equal to or more than the prescribed number of times in Step S108 is updated as a new zero point Z. Then, by using the measurement average Wa obtained in Step S110 and the updated zero point Z, a difference between the measurement average Wa and the zero point Z is calculated, and this calculated value is determined as a weight value and stored together with time.

That is, for judgment that “the animal has gotten onto the weighing pan 27,” the threshold A (full-side threshold) is set, and when a state where the measurement value is equal to or more than the threshold A continues for a certain period of time (for example, 1 second or more), the animal is judged to have gotten onto the weighing pan. Just after the start of the experiment, the threshold A is set based on a known weight of the animal, such as a value estimated from weight measurement before the experiment, or a value roughly grasped through a plurality of measurements, however, after the experiment is started and a plurality of measurement values are acquired, the threshold A is updated with time based on an average. More preferably, as the threshold A, an upper limit and a lower limit that are ±β% of an average value of the weight (for example, ±2 to 10% of the average value) are set, and a state where the measurement value is not less than the lower limit and not more than the upper limit, is judged as having gotten onto the weighing pan. Accordingly, oscillation of the center of gravity of the animal can be allowed.

On the other hand, that “the animal has gotten off the weighing pan 27” is judged when a state where the measurement value is less than the threshold A (or less than the lower limit A2) continues for a certain period of time (for example, 1 second or more) in principle. As in the flowchart described above, when no animal is on the weighing pan 27, based on a measurement value stable on the zero side, a threshold B (threshold on the zero side) for judgment that the animal has gotten off may be set. That is, by judging that the animal has gotten off when the state where the measurement value is not more than the threshold B continues for a certain period of time, erroneous measurement in a case where a measurement value becomes stable at a value less than the threshold A due to a state where a half of the body of the animal is on the weighing pan or a state where a tail of the animal touches the breeding container 22, can be avoided.

By obtaining (determining) the weight in this way, no matter when the animal gets onto the weighing pan 27, even when the animal moves on the weighing pan 27, and even when an object other than an animal, such as excrement, feed, etc., is placed on the weighing pan 27, an accurate weight can be measured.

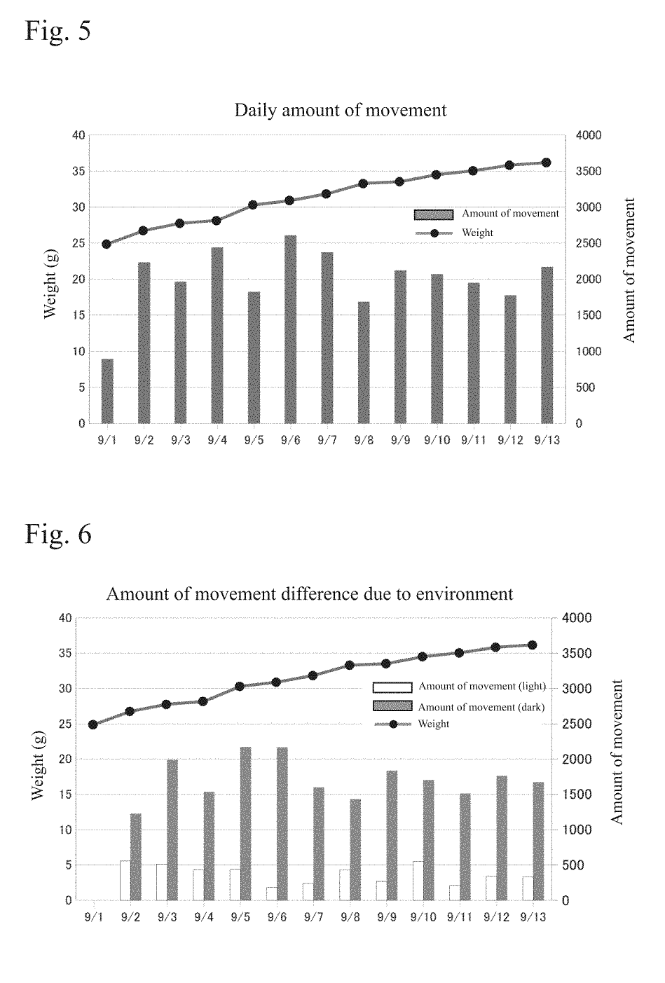

Next, FIG. 5 and FIG. 6 show preferable examples in which an amount of movement obtained as described above is visualized and output, and which are examples output in Step S9 of FIG. 3.

FIG. 5 shows a result of measurement of a mouse having an initial weight of 25.0 g for 13 days according to the flowcharts of FIG. 3 and FIG. 4, and the horizontal axis shows time (day), the left vertical axis shows weight (g), and the right vertical axis shows amount of movement (-). The sequential measurement was made by measuring the measurement value once per 0.1 seconds and setting the calculation interval of the amount of movement t to 24 hours. It could be confirmed that an amount of movement of an animal could be quantitatively measured by the system 1.

FIG. 6 shows a result of measurement of an amount of movement of a mouse having an initial weight 25.0 g for 12 days according to the flowcharts of FIG. 3 and FIG. 4 while the breeding room was switched to be light/dark every half day, and the horizontal axis shows time (day), the left vertical axis shows weight (g), and the right vertical axis shows amount of movement (-). Sequential measurement was performed in a state where the measurement value was measured once per 0.1 seconds, and the calculation interval of the amount of movement t was set to 12 hours. The colorless bar graph shows the time when it is light, and the colored bar graph shows the time when it is dark. A mouse is nocturnal, so that the amount of movement is larger at the time when it is dark. Such a change in amount of movement due to the environment could be quantitatively confirmed by the system 1.

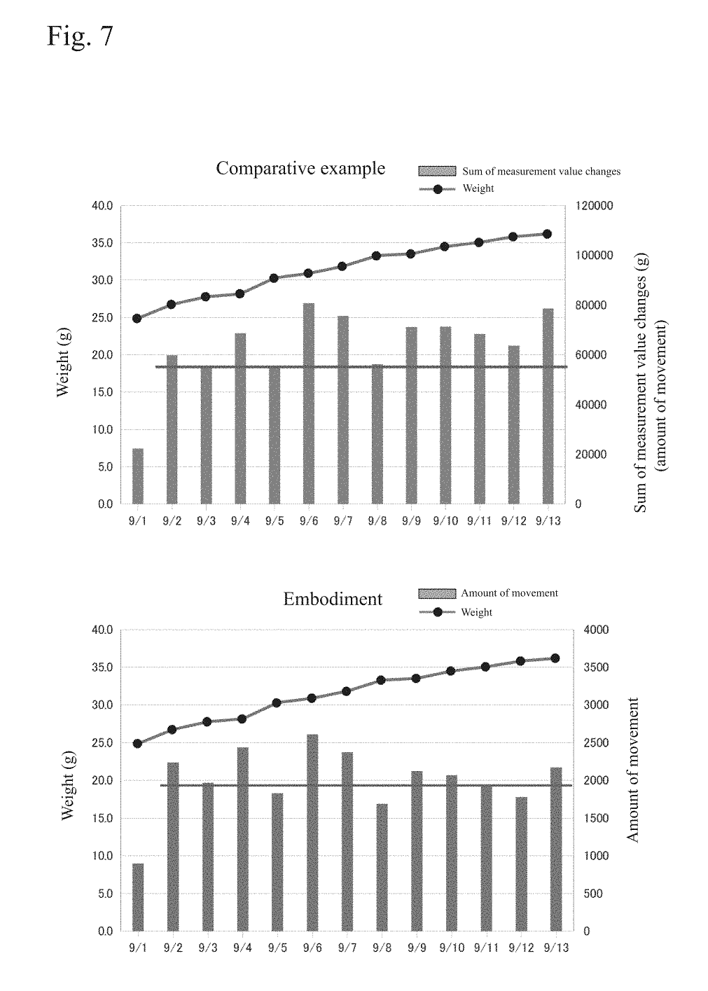

FIG. 7 shows comparison of the graph of FIG. 5 (the lower graph in FIG. 7) with a comparative example (the upper graph in FIG. 7). The comparative example (the upper graph in FIG. 7) is a graph in which a measurement value that is the same as the measurement value used in the graph of FIG. 5 is used, however, a value before dividing the sum of measurement value changes by the weight (that is, Steps S1 to S4 of FIG. 3 are performed, and an integrated value S in Step S4 is defined as an amount of movement and is not subjected to “division of the integrated value S by the weight value” in Steps S7 and S8) is output.

For comparison, an auxiliary line is drawn at the position of the weight of 20 g. During this experiment, the weight of the mouse increased from 25 g to 36 g, so that in the comparative example (the upper graph in FIG. 7), it can be confirmed that the amount of movement tends to increase along with the increase in weight. As the weight increases, the measurement value change becomes larger although the motion of the mouse is the same. On the other hand, in the system 1 (the lower graph in FIG. 7), even when the weight changes, an amount of movement is evaluated as the same as long as the motion is the same, and the tendency of an increase in daily amount of movement along with the increase in weight is not observed.

As described above, according to the measurement method of the present embodiment, an amount of movement of an animal can be measured with a new technique, and regardless of the time of day or night, influences of factors including external stimuli such as light and sound, gender, age in weeks, and heredity, and dose of a medical agent or toxic agent on an amount of movement can be quantitatively measured. Such an amount of movement is calculated by utilizing a measurement value change associated with activity of each living body and a weight of the living body at that time, so that even if the weight of the animal increases or decreases during measurement, it becomes possible to compare motion quantities of mice with different weights. An amount of movement obtained in this way is visualized and output, so that comparison and analysis, etc., thereof can be easily performed.

In addition, as a measurement value to be used for calculation of the amount of movement, a measurement value acquired while the breeding environment is maintained is preferably used because the possibility of an influence of a measurement value change caused by an element unnecessary for the experiment on the amount of movement is reduced. In addition, as a weight to be used for calculation of the amount of movement, a weight determined by a difference between a measurement value when an animal gets onto the weighing pan and a measurement value when the animal gets off the weighing pan is preferably used because even when the weight of the animal increases or decreases with time or even when a foreign matter such as water or feed, etc., is placed on the weighing pan and changes the breeding environment, an amount of movement utilizing a value from which an influence of such a change is subtracted as necessary is obtained. For these reasons, an amount of movement can be measured with high accuracy for hours and for a long period of time.

Also, by the method of measuring an amount of movement of an animal according to the present invention, even with a configuration other than the animal weighing scale 2 used in the embodiment, that is, even by using a measurement value obtained by using a balance with a conventional structure in which the weighing pan is not disposed inside a breeding container, an amount of movement can be obtained. Similarly, even with a weight obtained by a method other than the method of measuring a weight of an animal used in the embodiment, that is, even by using an “actually measured weight” obtained by placing an animal taken out of the breeding container on the weighing pan as in the conventional case, an amount of movement can be obtained.

Preferred embodiments of the present invention are described above, and each embodiment and each modification can be combined based on knowledge of a person skilled in the art, and such a combined embodiment shall be included in the scope of the present invention.

### REFERENCE SIGNS LIST

1 Animal weight measurement system
2 Animal weighing scale
3 Analyzation equipment
21 Scale
22 Breeding container
27 Weighing pan
41 Analyzation unit
43 Display unit

1. (canceled)

2. A method of measuring an amount of movement of an animal comprising:
sequentially measuring a measurement value of an animal as a measuring target by a scale;
setting a predetermined calculation interval of the amount of movement;
calculating a difference between a latest measurement value and a previous measurement value;
calculating an integrated value by integrating absolute values of the differences; and
calculating, at the calculation intervals of the amount of movement, a value by dividing the integrated value by an average value of weights of the animal in the calculation interval of the amount of movement or a weight of the animal at a point of time in the calculation interval of the amount of movement, and defining the value as an amount of movement of the animal.

3. The method of measuring an amount of movement of an animal according to claim 2, wherein the amount of movement is visualized and output by taking a time of calculation of the amount of movement on one axis and the amount of movement on the other axis.

4. The method of measuring an amount of movement of an animal according to claim 2, wherein as the measurement value, a value acquired in a state where a weighing pan of the scale is disposed inside a breeding container of the animal is used.

5. The method of measuring an amount of movement of an animal according to claim 2, wherein a weight of the animal is determined by a difference between a measurement value when the animal is judged to have gotten onto the weighing pan and a measurement value when the animal is judged to have gotten off the weighing pan.
